(19) United States
(12) Patent Application Publication
(10) Pub. No.: US 2003/0068286 A1
(43) Pub. Date: Apr. 10, 2003

(54) Self-tanning compositions having improved coloration using 3,5-dimethoxy-4-hydroxybenzaldehyde and 1,3-dihydroxyacetone

(76) Inventor: Eric Matthew Stroud, Oak Ridge, NJ (US)

Correspondence Address: ERIC M. STROUD, 146 NOTCH ROAD, OAK RIDGE, NJ, 07438, US

(21) Appl. No.: 10/263,575

(22) Filed: Oct. 3, 2002

Related U.S. Application Data
(60) Provisional application No. 60/327,163, filed on Oct. 5, 2001.

(51) Int. Cl. A61K 7/42

(52) U.S. Cl. 424/59

(57) ABSTRACT

The undesirable orange-brown coloration in the skin resulting from degraded 1,3-dihydroxyacetone self-tanning compositions is corrected by producing an olive-green coloration in the skin. A golden-brown artificial tan coloration is achieved using 3,5-dimethoxy-4-, which produces an olive-green colored melanoidins in the upper layers of the skin, and 1,3-dihydroxyacetone, which produces an orange-brown melanoidins in the upper layers of the skin. The visual resultant of the olive-green and orange-brown melanoidins is a natural-looking, golden-brown artificial tan coloration. The 3,5-dimethoxy-4-hydroxybenzaldehyde may also be used as a corrective composition, to adjust the coloration of previously applied 1,3-dihydroxyacetone-induced artificial tans.

### CROSS REFERENCE TO RELATED APPLICATIONS

This application claims the filing benefit under 35 U.S.C. § 119(e) of U.S. Provisional Application 60/327,163, filed Oct. 5, 2001, included herein by reference.

### TECHNICAL FIELD

The present invention is utilizes 3,5-dimethoxy-4-hydroxybenzaldehyde and 1,3-dihydroxyacetone to improve the coloration produced by the Maillard reaction, or “self-tanning” mechanism. The 3,5-dimethoxy-4-hydroxybenzaldehyde produces an olive-green in the skin while the 1,3-dihydroxyacetone produces an orange-brown coloration in the skin. Once applied together to the skin, the resultant coloration has the appearance of a natural golden brown tan.

### BACKGROUND OF THE INVENTION

Cosmetic and pharmaceutical formulations that utilize 1,3-dihydroxyacetone, or “DHA”, typically suffer from off-color tanning when stored for long periods of time. Pure DHA, in its dimerized form, must be kept refrigerated during storage in order to prevent chemical decomposition. Once included in a formulation, DHA is sensitive to functional nitrogen compounds, such as amines, as well as metal oxides, bases, acids, and reactive water-soluble compounds. Over time, certain ingredients in a cosmetic formulation chemical degrade the DHA, which is usually indicated by a decrease in pH. High temperatures increase the reaction rate of the degradation of DHA. When degraded DHA formulations are applied to the skin, an orange coloration results, instead of a natural brown tan coloration.

The present invention solves the coloration instability problems of DHA by combining a secondary reactive compound that also induces a Maillard reaction in the skin. The secondary compound, 3,5-dimethoxy-4-hydroxybenzaldehyde, is applied to the skin along with DHA. The 3,5-dimethoxy-4-hydroxybenzaldehyde produces olive-green colored melanoidins in the upper layers of skin, while the DHA produces orange-brown melanoidins in the upper layers in the skin at approximately the same reaction rate of the 3,5-dimethoxy-4-hydroxybenzaldehyde. The combined coloration, orange-brown and olive-green, results in a golden brown color, producing a more natural-looking artificial tan.

### SUMMARY OF THE INVENTION

Other features and advantages of the present invention will become apparent from the following detailed description, taken in conjunction with the accompanying drawings, which illustrate, by way of example, the principles of the invention.

The present invention is directed to the use of the combination of DHA and 3,5-dimethoxy-4-hydroxybenzaldehyde in order to produce a natural-looking artificial tan in the skin.

Over time, the chemical stability of unprotected DHA is reduced, producing undesirable chemical compounds such as methylglyoxal and formic acid. Degraded, or “stale” DHA produces an orange coloration on the skin once applied to the skin. Unfortunately, these results are not immediately known. The Maillard reaction produced by the DHA take sup to 5 hours to produce melanoidins, or colored compounds, in the upper layers of the skin. By the time an undesirable coloration develops, the wearer is helpless to remove or adjust the coloration, and must wait until their upper layers of skin are shed in order to rid themselves of the off-color.

A chemical compound has been found that is chemically compatible with DHA, which also produces an undesirable coloration in the skin using the same Maillard reaction mechanism as DHA. This compound, 3,5-dimethoxy-4-hydroxybenzaldehyde, or “syringaldehyde”, is a reactive aldehyde that produces olive-green melanoidins in the upper skin layers. While very undesirable to be used alone, the olive green coloration serves to balance the orange coloration provide by DHA. Thus, the combination of olive-green and orange-brown melanoidins in the upper skin layers presents and appearance of a golden brown to the human eye.

The Maillard reaction of the 3,5-dimethoxy-4-hydroxybenzaldehyde and the DHA proceed at approximately the same reaction rate, with 3,5-dimethoxy-4-hydroxybenzaldehyde being slightly slower because of its larger molecular size and longer penetration time into the skin. The reaction rates can be balanced by utilizing a skin penetration enhancement agent, such as a glycol ether, to more effectively deliver the 3,5-dimethoxy-4-hydroxybenzaldehyde to the upper skin layers.

The 3,5-dimethoxy-4-hydroxybenzaldehyde, as a water-insoluble aldehyde, is chemically compatible with water-soluble DHA. The compounds exist in two separate phases of a cosmetic formulation, the water phase and the oil phase. The 3,5-dimethoxy-4-hydroxybenzaldehyde may also be applied after DHA has been applied to the skin, in the event that an unexpected orange coloration develops and must be adjusted to back to a golden brown.

The concentration of 3,5-dimethoxy-4-hydroxybenzaldehyde with respect to DHA in a composition containing both should be adjusted to achieve the proper coloration. For compositions that are prone to DHA degradation, 3,5-dimethoxy-4-hydroxybenzaldehyde should be used at the same weight percent as the DHA. In compositions where the DHA is protected or stabilized, the 3,5-dimethoxy-4-hydroxybenzaldehyde should be used in relatively smaller amounts by weight than the DHA.

According to one aspect, the invention is the use of 3,5-dimethoxy-4-hydroxybenzaldehyde and DHA to produce a gold brown artificial tan in the skin.

According to another aspect, the present invention is the use of 3,5-dimethoxy-4-hydroxybenzaldehyde to adjust the undesirable orange-brown artificial tan coloration produced by DHA in the skin.

According to yet another aspect, the present invention is the use of 3,5-dimethoxy-4-hydroxybenzaldehyde and DHA for self-tanning compositions with improved coloration.

Further details of the above-described method and system are set forth below in the discussion of the preferred embodiments, which should be read in conjunction with the accompanying drawings. As will be apparent from the description below, the present invention and its variations are suitable for use with any self-tanning formulations which utilize 3,5-dimethoxy-4-hydroxybenzaldehyde and DHA.

### BRIEF DESCRIPTION OF THE DRAWINGS

FIG. 1 illustrates the chemical structure of 3,5-dimethoxy-4-hydroxybenzaldehyde.

FIG. 2 describes a self-tanning composition containing both 3,5-dimethoxy-4-hydroxybenzaldehyde and DHA.

FIG. 3 details a self-tanning composition containing 3,5-dimethoxy-4-hydroxybenzaldehyde that is applied to correct orange-brown coloration cause by a previous application of degraded DHA.

### DETAILED DESCRIPTION OF THE INVENTION

The instant invention relates to the use of 3,5-dimethoxy-4-hydroxybenzaldehyde and DHA to produce natural looking artificial tan. More particularly, the present invention is directed to, and concerned with, improving the coloration of artificial tans through the use of 3,5-dimethoxy-4-hydroxybenzaldehyde and DHA in self-tanning compositions.

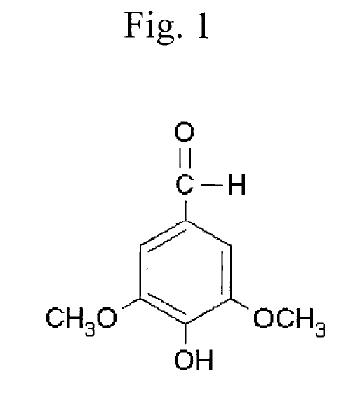

FIG. 1 illustrates the chemical structure of 3,5-dimethoxy-4-hydroxybenzaldehyde. Structurally, this compound is a vanillin, or a hydroxymethoxybenzaldehyde. The combination of the two methoxy groups, hydroxyl function, and aldehyde function give syringaldehyde is ability to produce coloration. Similar vanillins, such as vanillin, ethylvanillin, and ortho-vanillin do not produce similar coloration or any Maillard reaction mechanism.

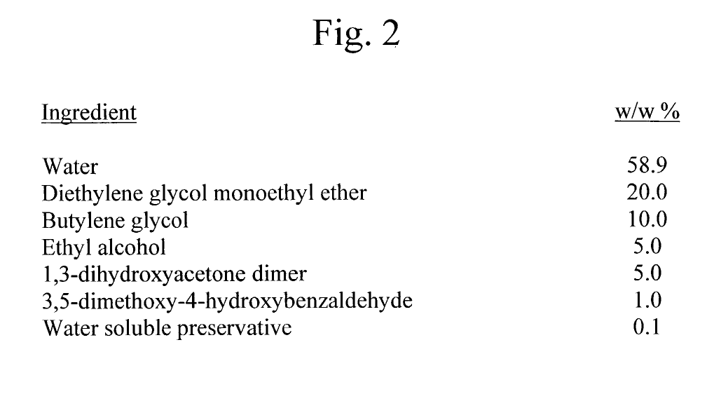

FIG. 2 describes a self-tanning composition containing both 3,5-dimethoxy-4-hydroxybenzaldehyde and DHA. This composition is a self-tanning spray, which is applied to the skin using a pump spray actuator or a bladder-type aerosol. Nitrogen functions, strong acids, strong bases, and metal oxides are absent in order to preserve the stability of the DHA. The composition is all within an aqueous phase, where glycols and glycol ethers serve to solvate the 3,5-dimethoxy-4-hydroxybenzaldehyde.

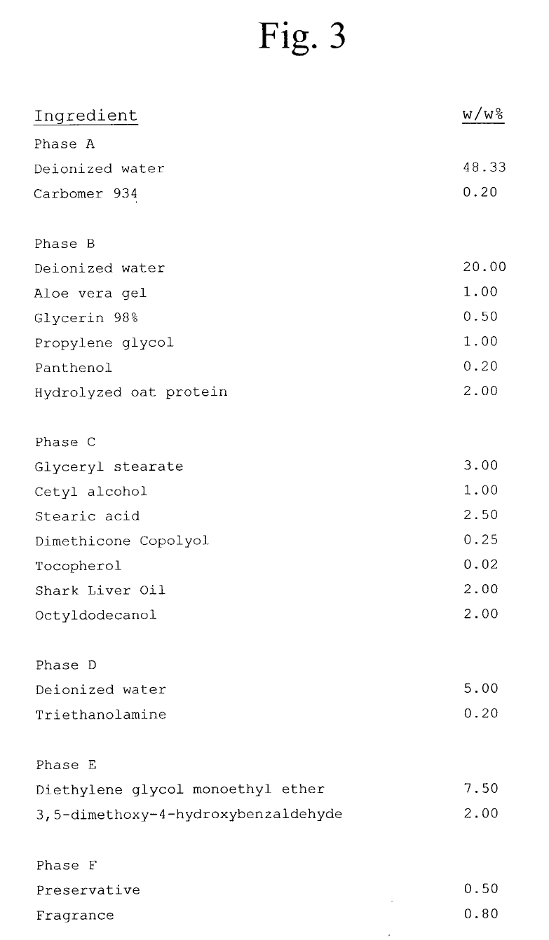

FIG. 3 details a self-tanning composition containing 3,5-dimethoxy-4-hydroxybenzaldehyde that is applied to correct orange-brown coloration cause by a previous application of degraded DHA. This composition is an oil-in-water emulsion which is applied from a tube or bottle as a lotion. The lotion is applied to areas of the skin which have developed poor coloration resulting from DHA. The 3,5-dimethoxy-4-hydroxybenzaldehyde is added to the composition in Phase E, to ensure that high temperature and the neutralization of the carbomer does not degrade or alter the 3,5-dimethoxy-4-hydroxybenzaldehyde's functionality. This formulation is prepared by separately premixing Phases B, C, D, E, and F. Phase E should be premixed using high shear. All of the Phase A water is loaded into a mixing vessel and heating the water to 65° C. The carbomer is slowly sifted into the heated water with high agitation, and is mixed until uniform. Phase B is added to Phase A under moderate mixing speed. Phase C ingredients are heated and premixed at 78° C. While Phase A-B is held at 75° C., Phase C at 78-80° C. is added to Phase A under moderate mixing. Heat is removed from the batch. When the batch temperature reaches 65° C., Phase D is added under slow mixing. The batch is cooled to 30° C., and Phases E and F are slowly added under slow mixing. The cosmetic vehicle is then packaged.

I claim:

1. A composition that is applied to the skin having improved artificial tan coloration comprised of:
(a) A concentration between 0.01% and 10% by weight of 1,3-dihydroxyacetone; and,
(b) A concentration between 0.01% and 10% by weight of 3,5-dimethoxy-4-hydroxybenzaldehyde.

2. A composition having improved artificial tan coloration containing at least 0.01% by weight of 3,5-dimethoxy-4-hydroxybenzaldehyde that is applied to the skin

3. Said composition in claim 2 is applied after a previous application of 1,3-dihydroxyacetone to the skin.

4. A composition containing at least 0.01% by weight of 3,5-dimethoxy-4-hydroxybenzaldehyde that is applied to the skin in order to produce an olive green coloration.
